(19) United States
(12) Patent Application Publication
(10) Pub. No.: US 2010/0003754 A1
(43) Pub. Date: Jan. 7, 2010

(54) METHODS OF DIFFERENTIATING STEM CELLS

(75) Inventors: Arne Briest, Karlsruhe (DE); Steen Sindet-Pedersen, London (GB)

Correspondence Address: IP GROUP OF DLA PIPER LLP (US), ONE LIBERTY PLACE, 1650 MARKET ST, SUITE 4900, PHILADELPHIA, PA, 19103, US

(73) Assignee: Ossacur AG, Oberstenfeld (DE)

(21) Appl. No.: 12/373,757

(22) PCT Filed: Sep. 13, 2007
(86) PCT No.: PCT/EP07/07971

§ 371 Date: Feb. 5, 2009

(30) Foreign Application Priority Data
Sep. 15, 2006 (DE) 10 2006 045 848.6
Dec. 13, 2006 (DE) 10 2006 060 247.1

(51) Int. Cl. C12N 5/00

(52) U.S. Cl. 435/377; 435/325

(57) ABSTRACT

A method for at least partial differentiating stem cells and/or progenitor cells to at least one tissue type includes treating the stem cells and/or the progenitor cells with extracts which include active substances and/or components for differentiating stem cells and/or progenitor cells, and cultivating and differentiating the stem cells and/or the progenitor cells.

### RELATED APPLICATIONS

This is a §371 of International Application No. PCT/EP2007/007971, with an international filing date of Sep. 13, 2007 (WO 2008/031588 A1, published Mar. 20, 2008), which is based on Gernan Patent Application Nos. 102006045848.6, filed Sep. 15, 2006, and 102006060247.1, filed Dec. 13, 2006.

### TECHNICAL FIELD

This disclosure relates to methods for the at least partial differentiation of stem cells and/or progenitor cells to at least one tissue type, extracts provided for this purpose, and methods for the preparation thereof from organic materials. The disclosure also relates to uses of the extracts for the differentiation of stem cells and/or progenitor cells, and the uses thereof for the therapy of tissues.

### BACKGROUND

In the context of techniques for tissue cultivation, so-called “tissue engineering,” relating to a treatment of organ damage, especially an improvement or restoration of biological functions, there is intensive discussion about the use of stem cells.

Conventional differentiation techniques show, because they have not been fully developed, considerable disadvantages relating to the differentiation both of stem cells and progenitor cells. It is necessary to maintain complicated conditions for the methods for cell differentiation.

To obtain tissues in practice, frequently aggregates of embryonic stem cells, called embryoids, are used to differentiate the latter into desired tissue types. In some countries, for a number of reasons, especially ethical considerations, it is not possible to use embryoids or stem cells extensively for cell differentiation.

A further disadvantage is that desired differentiation results can be achieved only conditionally with adult stem cells obtained from conventional methods. “Effects of bone protein extract on human mesenchymal stem cells proliferation and differentiation” by C. Woo et al. describes a study of the differentiation of mesenchymal stem cells with the aid of a bovine bone extract. This revealed a certain effect of the extract on the proliferation and differentiation activity of stem cells. However, further successes would be very desirable in particular for the treatment of serious disorders such as myocardial infarction, blood cancer, amyotrophic lateral sclerosis (ALS), Alzheimer's or multiple sclerosis.

There has been criticism of animal experiments, for example for cosmetic, hygiene or medical research. However, it is at present possible only with difficulty to comply with demands for a shift to experiments in cell cultures. There is thus intensive research in the area of tissue cultivation to be able to offer alternatives in the form of tissue cultures with which the experiments can be carried out in Petri dishes. There is thus an increased demand for techniques for producing particular tissue types and, associated therewith, for techniques for stem cell cultivation and differentiation.

It could therefore be helpful to provide improved methods for the proliferation and differentiation of stem cells and/or progenitor cells in cell culture, especially for tissue cultivation.

### SUMMARY

We provide a method for at least partially differentiating stem cells and/or progenitor cells to at least one tissue type, including treating the stem cells and/or the progenitor cells with extracts which include active substances and/or components for differentiating stem cells and/or progenitor cells, and cultivating and differentiating the stem cells and/or the progenitor cells.

We also provide a method of producing extracts in which organic materials are subjected to at least one denaturation operation for extraction including providing cells and/or tissues as organic materials which include active substances and/or components which activate or inhibit growth and/or differentiation of stem cells and/or progenitor cells to at least one tissue type.

We further provide an extract produced according to the method for at least partially differentiating stem cells and/or progenitor cells to at least one tissue type, including active substances and/or components (which are not derived from bone) which activate or inhibit the growth and/or differentiation of stem cells and/or progenitor cells to at least one tissue type.

We still further provide a method for differentiating stem cells and/or progenitor cells including subjecting undifferentiated cells and/or incompletely differentiated cells to a treatment with extracts wherein the extracts have been obtained from cells and/or tissues.

### BRIEF DESCRIPTION OF THE DRAWINGS

FIG. 1 is a graph of the number of colonies per given number of cells based on colony type in the presence of bone marrow extract (HSC002) and in the absence of bone marrow extract.

FIG. 2 is a graph of the number of colonies per given number of different cells based on colony type in the presence of bone marrow extract (HSC003) and in the absence of bone marrow extract.

FIG. 3 is a graph of the number of colonies per given number of different cells based on colony type in the presence of bone marrow extract (HSC004) and in the absence of bone marrow extract.

FIG. 4 is a graph of the number of colonies per given number of different cells based on colony type in the presence of bone marrow extract (COLLOSS® E) and in the absence of bone marrow extract.

FIG. 5 is a graph of the number of colonies per given number of different cells (BFU-E) based on colony type in the presence of bone marrow extract (HSC003) and in the absence of bone marrow extract.

FIG. 6 is a graph of the number of colonies per given number of different cells (CFU-M) based on colony type in the presence of bone marrow extract (HSC004) and in the absence of bone marrow extract.

FIG. 7 is a graph of the number of colonies per given number of different cells (BFU-E) based on colony type in the presence of bone marrow extract (HSC005) and in the absence of bone marrow extract.

FIG. 8 is a graph of the number of colonies per given number of different cells based on colony type in the presence of bone marrow extract (HSC004+HSC005/HSC003+HSC005) and in the absence of bone marrow extract.

### DETAILED DESCRIPTION

We provide methods for the at least partial differentiation of stem cells and/or progenitor cells to at least one tissue type, comprising a treatment of the stem cells and/or of the progenitor cells with extracts which include active substances and/or components for the differentiation of stem cells and/or progentiro cells, and a cultivation and differentiation of the stem cells and/or of the progenitor cells.

“Stem cells” mean undifferentiated cells or substantially undifferentiated cells of the same cell line. Such stem cells are able to differentiate and in this way able to replace cells lost by an organism, especially dead or mutated cells. The stem cells are able to produce continuously unchanged daughter cells which have a latent capability for differentiation. The stem cells therefore show a characteristic capability of self-renewal. In addition, the stem cells also have the capability of producing daughter cells which show stronger or different differentiations. In other words, stem cells show the capability of passing through cell cycles, and in this way repeatedly generating at least one daughter cell which has a comparable endowment to its parent cell, and which is able to differentiate exactly like its partent cell.

“Progenitor cells” is intended to include, in addition to the conventional meaning, also putative stem cells for which self-renewal has not to date been proven.

Every stem cell, in particular also stem cells unknown to date, is suitable for treatment with our methods.

It is possible to use, for example, embryonic, fetal, neonatal, adult (somatic), especially bone marrow-derived, preferably mesenchymal, hematopoietic stem cells and/or progenitor cells.

“Neonatal stem cells” mean in particular stem cells from umbilical cord blood.

To be able to undertake a classification of stem cells, they are usually divided according to their capability of differentiation. “Totipotent stem cells” means stem cells which are able to form complete organisms. “Pluripotent stem cells” means stem cells able to form every cell line of the organism, including germ cells. Embryonic stem cells should be mentioned as an example thereof. “Multipotent stem cells” means stem cells able to form a plurality of cell lines from which complete tissues are formed. Hematopoietic stem cells should be mentioned as example thereof. “Oligopotent stem cells” means stem cells able to form at least two cell lines of a tissue. Neuronal stem cells able to form subclasses of neurons in the brain should be mentioned as example thereof. “Unipotent stem cells” means stem cells able to form a single cell line. Spermatogonal stem cells should be mentioned as an example thereof.

“Tissue” includes every tissue formed from cells. “Tissue” also includes particular organs. “Organs” means parts of the bodies of organisms, especially of vertebrates, preferably of human origin. Organs also means functional units composed of various tissues. Examples of tissues are bone, cartilage, muscle, especially cardiac or skeletal muscle. Further tissues which should be mentioned are in particular meniscus, tendon, ligament, lung, blood, skin, liver, kidney, glandular tissue, aorta, pancreas, brain and/or nerve tissue.

In one aspect of our methods, embryonic, fetal, neonatal, adult (somatic), especially bone marrow-derived, preferably mesenchymal, hematopoietic stem cells and/or progenitor cells are used for the treatment.

In a further aspect of our methods, the stem cells and/or progenitor cells exhibit, after treatment with the extracts, on their cell surface specific molecules, in particular specific molecules for cells of particular tissues, and/or a morphology characteristic of particular tissues.

The stem cells treated with the extracts advantageously develop on their cell surface specific molecules, in particular molecules specific for particular tissue types. The specific molecules can be detected with the aid of standard methods. For example, detection by antibodies, especially labeled antibodies, preferably fluorescence-labeled antibodies, is possible. The specific molecules are preferably various membrane surface proteins, in particular CDIOS (endoglin), CD133 (hprominin), CD271 (LNGFR) and CD45, preferably STRO-1. A presence or absence of such membrane surface proteins provides the possibility of separating different cell populations, and undertaking an estimation of whether the differentiation into the desired tissues has taken place.

In a further aspect of our methods, extracts derived from organic materials are employed for the treatment.

“Organic materials” means materials, especially tissues, which are of human, animal, vegetable, microbial origin, and combinations in the form of mixed materials.

“Animal” or “of animal origin” includes in particular all vertebrates, preferably mammals.

Various experiments on the symmetrical/asymmetrical cell division of stem cells proves that stem cells and progenitor cells are decisively influenced in their self-renewal or differentiation behavior by their environment. Dependencies of proliferation and the differentiation of cells is known, for example, for wound healings. Such a behavior of cells is referred to as niche behavior.

“Niche” means neighboring cells, extracellular matrix and the constituents thereof, and further parameters which occur in the natural environment and which influence the behavior of cells, especially the behavior of stem cells.

It has surprisingly been found that stem cells which are treated with our extracts develop a morphology which is characteristic of the tissues from which the extracts were obtained.

Extracts are represented in particular by the extracts COLLOSS® and COLLOSS® E which are produced and commercially distributed by the assignee and which include collagen respectively of bovine and equine origin and active substance complexes.

Extracts of this type which are in particular of natural origin advantageously simulate the niche for the stem cells. It has surprisingly been found that besides the formation of specific molecules on the cell surface and/or the formation of a characteristic tissue morphology, the differentiated stem cells produce characteristic substances of the tissues from which the extracts were obtained. The differentiation is proved through the formation of such substances. In particular, the production of so-called “differentiation markers” shows that a differentiation has taken place. Suitable for this purpose are substances like collagen, preferably collagen of type I, osteopontin, calcium, especially a deposition of calcium in the cells, and a formation of aggrecan. It is also possible to provide further investigation steps on the differentiated stem cells. It is also provided to investigate the differentiated stem cells produced in the manner according to the disclosure or the undifferentiated stem cells for characteristic transcription factors. Transcription factors suitable for this purpose are in particular octamer-4 (OCT-4), Nanog and members of the SOX family, in particular SOX2, preferably SOX9.

In a further aspect of our methods, extracts which are of human and/or animal origin are employed for the treatment.

In a further aspect of our methods, extracts which are of vegetable and/or microbial origin, in particular derived from cell cultures, are employed for the treatment.

In a further aspect of our methods, extracts which have been obtained from cells and/or tissues are employed for the treatment.

This measure has the advantage that further sources for the industrial production of the extracts can be opened up. It is possible to use tissues cultivated in cell culture to obtain the extracts. In this way, extracts can be obtained in large quantities which are continuously available.

In a further aspect of our methods, extracts which are derived from bone and/or cartilage, muscle, in particular cardiac or skeletal muscle, tendon, ligament, lung, blood, skin, liver, kidney, glandular tissue, brain and nerve tissue, and moreover are preferably of bovine and/or equine and/or human origin, are employed for the treatment. Natural sources suitable for obtaining the extracts employed are also meniscus, aorta and/or pancreas.

The methods can, as already described, be carried out with extracts which can be produced from any tissue. Preferred tissue types are indicated herein.

We also provide methods for producing extracts in which organic materials are subjected to at least one denaturation operation for the extraction, where cells and/or tissues are provided as organic materials which include active substances and/or components which activate or inhibit, in particular activate, the growth and/or differentiation of stem cells and/or progenitor cells to at least one tissue type.

Concerning the organic materials which can be employed and tissues which can be employed, reference is made to the description already given.

We provide for the cells and/or the tissues for producing the extracts to be processed, in particular purified, preferably defatted and/or comminuted. Various denaturants can be used for the denaturing. Examples of suitable denaturants to be mentioned are acids, alkalis and/or salts. Also, suitable are chaotropic substances, preferably guanidine and/or guanidinium hydrochloride, and/or detergents. A 4M guanidinium hydrochloride solution is particularly suitable. These denaturants can also be used in combination. It is further provided for the denaturants to be removed by denaturing steps. By this are meant in particular a wide variety of dialysis methods and chromatography methods known to a skilled worker and suitable for removing denaturants. The extracts produced by the method have advantageously passed through at least one renaturing step. This is advantageous to remove substances harmful for cells from the extracts. The extracts are thus prepared for further processing, and their composition is optimally produced in relation to their effect. In particular, an alternation of denaturing steps and subsequent renaturing steps is conceivable, preferably to enrich active substances in the extracts.

In a further aspect, the extracts produced are in the form of lyophilized extracts.

In particular, the product COLLOSS® or COLLOSS® E takes the form of lyophilized extracts of the type described herein, which can preferably be produced by our methods for producing the extracts and can be employed in the treatment of stem cells.

The extracts produced by our methods preferably include active substances and/or components which influence the growth and/or the differentiation of stem cells or progenitor cells. As already explained, it is extremely advantageous for a cultivation or differentiation of stem cells to be able to present an environment which is comparable to that encountered in natural, normally functioning tissues. Stem cells and/or progenitor cells are advantageously stimulated by such extracts to differentiate into the tissue types from which the extracts have been produced. It is particularly important in this connection that, besides activating or inhibiting active substances, there is provision of components which are helpful for simulating the natural environment.

It is particularly possible, as in the case of the products COLLOSS® and COLLOSS® E, for the structures to differ in distinctiveness depending on the tissue type. This particularly relates to collagen, preferably a collagen of type I which, besides individual α-collagen molecules, includes further structures in the form of superstructures. The extracts produced by our methods are advantageously to be categorized as biologically acceptable. It is therefore possible for the cells treated with the extracts theoretically to be employed immediately after their treatment, especially with exclusion of further processing, directly for their desired use.

In a further aspect of our methods for producing extracts, the organic materials take the form of cartilage, muscle, in particular cardiac or skeletal muscle, tendon, ligament, lung, blood, skin, liver, kidney, glandular tissue, brain or nerve tissue, preferably of bovine, equine and/or human origin. Preferred organic materials in this connection are also meniscus, aorta and/or pancreas.

In a further aspect, the extract produced by our processes comprises active substances and/or components (which are not derived from bone) which activate or inhibit, in particular activate, the growth and/or differentiation of stem cells and/or progenitor cells of at least one tissue type.

The tissues determine which active substances and/or components are included in the extracts. It is also provided for additionally further active substances and/or components to be added to those already present in the extracts. This is useful in particular for precise regulation of the growth and/or differentiation of the stem cells and/or of the progenitor cells. This is used preferably for deliberate production of particular subtypes of tissues.

In a further aspect of our methods for producing extracts, the active substances and/or the components comprise at least leukotrienes, cytotactin, tenascin, laminin, fibronectin, cytokines, especially osteogenic active substances, preferably BMP-I, BMP-II, IGF1, TGF-β1, FGF and PDGF, collagen, in particular of type I.

Denaturation of natural or native components of the extracts disrupts their native three-dimensional structures, and a subsequent restructuring of the components can take place. Depending on the chosen intensity of a denaturation operation and depending on the chosen denaturant, some parts of the components are more affected by the denaturation than others, and where appropriate some parts of the components are not affected at all. However, a complete denaturation can also be carried out. Through subsequent removal of the denaturant, for example by dialysis and/or chromatography, further structures of the components develop and influence the properties of the extracts, and differ from the native structure. It is particularly advantageous that the compact three-dimensional structure of the components is deranged or destroyed by denaturation and renaturation, and an in particular mesh-like structure is developed. The surface area of the components is enlarged in this way. Active substances concealed inside the components reach the surface thereof and are more accessible to cells. The enlargement of the surface area also in particular makes diffusion of the active substances out of the components easier.

It is further provided for the extracts to be modified in their properties. It is provided in particular for the extracts to be produced or provided with active substances which activate or stimulate and/or enhance later differentiation of cells. The active substances of the extracts are in this connection preferably of natural origin, i.e., the active substances are preferably in their native form. The extract advantageously comprises so-called “recruitment factors,” especially chemotactic agents (chemotaxins), for example leukotrienes, which act specifically on stem cells, preferably mesenchymal stem cells and/or cartilage progenitor cells. The active substances preferably also include substances which are known to be members of the family of adhesion factors, for example cytotactin, tenascin, laminin and/or fibronectin. Also used as active substances of the extracts are preferably growth factors and/or maturation factors, preferably cytokines, for the proliferation and differentiation of the cells. The growth factors are preferably bone growth factors, for example BMP (bone morphogenetic proteins), preferably BMP-2, BMP-7 and/or BMP-4, and IGF (insulin-like growth factor), preferably IGF1, and TGF (transforming growth factor), preferably TGF-β1. It is additionally or alternatively possible for FGF (fibroblast growth factor) and PDGF (platelet derived growth factor) to be present as growth factors.

In a further aspect of the extracts produced by our methods, the active substances and/or components are present in combination, preferably in a complex.

An “active substance complex” means a combination of various active substances. It is particularly suitable to use the products COLLOSS® and COLLOSS®D as extracts for stimulating differentiation.

These active substances advantageously show overlapping active functions, so that a possible loss of activity of one or more active substances can be taken over or compensated by the other active substances present in particular in a complex. The extracts may furthermore exhibit additive or synergistic effects for the differentiation of stem cells, in particular for a differentiation of stem cells to bone and/or cartilage cells.

We also provide for the use of extracts produced by our methods for the differentiation of stem cells and/or progenitor cells. The extracts are preferably employed in the area of tissue engineering. Tissue engineering can be carried out in particular in vitro. For example, the extracts can be used for regenerating liver and/or heart tissue. It is likewise provided in particular for the extracts to be used for regeneration of nerve damage and/or for the regeneration of skin.

In one aspect, the extracts are employed for differentiating mesenchymal stem cells to at least one tissue type, in particular to bone and/or cartilage tissue. The use also relates to the differentiation of stem cells to progenitor cells. The mesenchymal stem cells are preferably derived from bone marrow.

In another aspect, we provide for differentiation of hematopoietic stem cells to hematopoietic progenitor cells. We also provide for the use of stem cells and/or progenitor cells for a treatment with extracts for the therapy of tissues.

Further features and advantages are evident from the following description of selected, representative examples. In this connection, the individual features may in each case be implemented on their own or in combination with one another.

Bovine Joint and Meniscus Extracts for Chondrocytic In Vitro Differentiation of Human Mesenchymal Stem Cells Obtained from Bone Marrow (Bone Marrow-Derived Mesenchymal Stem Cells, BMMSC)

Differentiation was induced by using two differently diluted solutions of the joint or meniscus extracts, 1:25 and 1:50. The method was monitored and investigated at various times by using techniques for “real time polymerase chain reaction” (real time-PCR), immunohistochemistry (IHC), enzyme-coupled immunoadsorption (Enzyme Linked Immmunosorbent Assay, ELISA) and chondro-specific staining.

### 1. Production of the Extracts

The extracts were produced by powdering tissues, preferably in the deep-frozen state, for example by using liquid nitrogen. Suitable tissues are cartilage tissue, in particular from joint and/or meniscus. The resulting tissue powder was defatted with a solvent, in particular with an organic solvent, for example with acetone. A demineralization was expediently carried out, in particular with acids, preferably with mineral acids such as hydrochloric acid. 0.6 molar hydrochloric acid was employed. It may additionally be expedient to carry out a treatment with chelating agents, in particular with ethylenediaminetetraacetate (EDTA) and/or tris(hydroxylmethyl)aminomethane (TRIS). The tissues were extracted with chaotropic substances, in particular with guanidine, preferably with guanidinium hydrochloride. The extracts obtained in this way were dialyzed, in particular against buffers, to remove the chaotropic substances. Collagen and active substances were isolated in a single step by extracting the tissues. The extracts were subsequently lyophilized by standard methods.

Before the lyophilization, the extracts can also be subjected to further purification steps. It is possible to employ for the purification in particular chromatographic methods such as high performance liquid chromatography (HPLC).

Also employed as extracts were COLLOSS® and COLLOSS® E. The lyophilized extracts were expediently taken up in buffers, for example in phosphate buffer, before use thereof. The solution was prepared by using 1 mg to 10 mg of dry material of the lyophilized extracts per ml of the buffer. It is also possible to use solutions of lower or higher concentration in experiments.

2. Isolation of Human Mesenchyrnal Stem Cells Obtained from Bone Marrow (BMMSC)

The BMMSC are primitive cells which occur in bone marrow approximately in the ratio of 1:100 000 relative to nucleated cells.

The BMMSC were isolated by using standard methods from patients whose consent was obtained. The cells were plated in 100 mm Petri dishes and incubated and kept in cultures at 37° C., 95% air, 5% carbon dioxide and 100% humidity.

### 3. Cell Morphology

The morphology of the cells was inspected by using differential interference contrast microscopy (DIC-light microscopy) on day 1, 3, 7 and 14 of the cultures. Changes in the morphology were documented.

### 4. Characterization of the BMMSC

A presence of cell surface markers, in particular of STRO-1, was used to confirm that the cell cultures included multipotent BMMSC populations. STRO-1 expressing cells were identified by immunofluorescence methods. 1.5×10⁴ BMMSC were seeded in a 12-well plate on sterile coverslips. The cultures were supplemented with a control medium which comprised Dulbecco's modified Eagle's medium (1000 mg of glucose/L, L-glutamine, NaHCO₃ and pyridoxine•HCl, Sigma Aldrich D6046) with 1% penicillin/streptomycin and 10% fetal bovine serum (FBS, (Gibco). After 36 hours, the cells were fixed with 70% ethanol and then washed three times with a phosphate buffer (phosphate buffered saline, PBS) (Cambrex). The cells were then incubated with monoclonal antibodies against STRO-1 (R&D Systems, Minneapolis, Minn.) with a concentration of 10 μg/ml at 37° C. for one hour. The primary antibodies were removed, and the cells were then washed three times with the PBS, and subsequently the cells were incubated for one hour with anti-mouse fluorescein isothiocyanate conjugated immunoglobulin M antibodies (anti-mouse (FITC)-conjugated IgM) (Molecular Probes) with a concentration of 1:1000 for one hour. The secondary antibodies were removed, and the cells were washed three times with the PBS. Each well was incubated with Hoechst (1 μg/ml) for 10 minutes. A negative control was carried out with the secondary antibody. After the incubation, the cells were washed, put on supports and inspected using a fluorescence microscope.

### 5. Immunoprecipitation of Transforming Growth Factor-β1 (TGF-β1)

100 μl portions of the joint or meniscus extract were immunoprecipitated using anti-TGF-βB1 monoclonal antibodies (Chemicon) for at least 18 hours. A 12% (sodium dodecyl sulfate, SDS) SDS polyacrylamide gel was loaded with the proteins, subjected to an electrophoresis and transferred to a polyvinylidene difluoride (PVDF) membrane (Western Blot). The blot was then incubated with anti-TGF-β1 monoclonal antibodies. Resulting bands were visualized by chemoluminescence (Santa Cruz, USA).

### 6. Preparation of Samples for Detections

The cells were trypsinized and then counted using trypan blue. 60×10³ or 15×10³ mesenchymal stem cells were seeded in a 25 cm² cell culture bottle or in a 12-well plate. Each vessel was charged with an appropriate amount of standard medium (control group) or a medium which contained the joint or meniscus extract (of bovine origin) in a dilute solution of 1:25 or 1:50. Recombinant TGF-β1 protein (R&D Systems) with a concentration of 10 ng/ml was used for chondrocytic differentiation of the BMMSC as positive control. Cell supernatants were collected and stored at −80° C.

### 7. In Vitro Cytotoxicity and Cell Proliferation

The extracts were investigated for their toxicity for the cultivated cells using “CytoTox 96 Non-Radioactive Cytotoxicity” (Promega). This detection method determines quantitatively the lactate dehydrogenase (LDH), a stable cytosolic enzyme which is released by cytolysis. The released LDH was determined in culture supernatants with a coupled enzymatic detection method using a conversion of a tetrasodium salt into a red formazan product. An absorbance was measured at a wavelength of 492 nm. Results were obtained 6 hours after a treatment with the extracts.

Investigations on the cell proliferation were carried out using “CellTiter 96 Non-Radio-active” (Promega). This is a method based on 3-(4,5-dimethylthiazol-2-yl)-2,5-diphenyltetrazolium bromide (MTT) which determines the property of metabolically active cells of converting a tetrazolium salt. For example, the conversion of pale yellow MTT by cellular dehydrogenases into the dark blue, water-insoluble formazan is proportional to the activity of the dehydrogenases and thus to the survival rate of the cells. The absorbance is measured at a wavelength of 595 nm. The cells were counted and seeded in a density of 104 in a 96-well round-bottom plate. The cell proliferation was determined on day 1, 3, 7 and 14. 100 μl were put in three wells in each case. To determine the background, cells were seeded in wells which contained only the medium. 15 μl of dye solution were put in each well. The cells were incubated at 37° C., 100% humidity and 5% CO₂ for 4 hours. Two 100 μl aliquots were then removed from each well and transferred into a 96-well plate. 100 μl of a stop solution were then added to each well. The cells were then incubated at 37° C., 100% humidity and 5% CO₂ for at least one hour. The absorbance was measured at the wavelength of 595 nm with a reference wavelength of 655 nm.

### 8. Quantification of Chondroitin Sulfate

The cells were washed with the PBS and then incubated with Alcian blue stain (1% Alcian blue in 3% strength acetic acid) for 20 minutes. The cells were decolorized by washing three times with 3% strength acetic acid and washing once with water. To quantify the stain which has been taken up, the cells were solubilized by shaking in 1% strength SDS solution at room temperature for 30 minutes and then heating at 90° C. for one hour. The absorbance was detected at the wavelength of 595 nm.

### 9. Histological Staining

### a) Toluidine Blue Staining

The cells were hydrated and then incubated with toluidine blue stain for two to three minutes (pH 2). Three consecutive washing steps with distilled water were carried out, after which the cells were dehydrogenated with 95% strength and then with 100% strength ethanol (rapid immersion 10 times to avoid fading of the stain). Each step was followed by cleaning with xylene twice for three minutes each time. The cells were then inspected on supports using a light microscope.

### b) Hematoxylin and Eosin (H&E) Stain

The H&E stain was used to assess the morphology of the cells and the presence of calcium deposits in the cultures. 14 days after treatment with the extracts, the cells were fixed with 70% strength ethanol, exposed to hematoxylin for 40 seconds, treated with acidic alcoholic solution for one second, washed with aqueous ammonia for five seconds, stained with eosin for ten seconds, washed with alcohol and put on a support. The cells were inspected using a light microscope.

### 10. Cartilage-Specific Differentiation Markers

### a) SOX9

Total cellular ribonucleic acid (RNA) was extracted using “NucleoSpin RNA II” from Macherey-Nagel. 1 μg of the RNA was amplified using a “one-step RT-PCR kit” (ABgene, UK). Real time (RT) PCR primers specific for collagen type I, SOX9, Aggrecan and glyceraldehyde-3-phosphate dehydrogenase (GAPDH) were used. Amplification of the so-called housekeeping gene GAPDH was used to verify the quality of the RNA and ensure uniform loading of resolving gels with the samples. A real time reverse transcription PCR was carried out using a LightCycler (Roche Diagnostic). The RT-PCR conditions included an RT step of 20 minutes at 61° C. to produce single-stranded cDNA from mRNA, followed by an inactivation of reverse transcriptase and denaturation at 95° C. for 30 seconds. 40 amplification cycles of one second at 95° C., of 15 seconds at 58° C. and of 13 seconds at 72° C. were carried out. Finally, a cooling step of 30 seconds at 40° C. was carried out.

### b) Aggrecan

The cells were seeded in a density of 15×10³ in a 12-well plate. 14 days after the treatment with the extracts, the cells were washed with PBS and then fixed with ice-cold 70% strength ethanol for at least 18 hours. The cells were then blocked in 3% strength goat serum (normal goat serum NGS) for one hour, and thereafter mouse anti-Aggrecan antibodies (Chemicon) were used with a concentration of 1 μg/ml and incubated at 4° C. for at least 18 hours. The primary antibodies were then removed, the cells were washed three times with PBS and subsequently incubated with anti-mouse FITC-conjugated IgM (Molecular Probes) in a ratio of 1:1000 for one hour. The secondary antibodies were removed and the cells were washed three times with the PBS. A negative control was carried out with the secondary antibody. After the incubation, the cells were washed, put on supports and inspected using a fluorescence microscope.

11. Analysis of the cellular supernatants

### a) Secretion of Collagen Type I

A collagen determination was carried out to check whether an increase in the collagen content results in the BMMSC cultures in the presence of joint and meniscus extracts. Acid-soluble collagen was determined in the culture media and cell lysates on day 14, using “sircol collagen assay” (Biocolor) in accordance with the manufacturer's information with collagen type I as standard.

### b) Secretion of Osteopontin

The osteopontin (OPN) secretion was carried out using a “humanen OPN TiterZyrne ELISA kit” (Assay designs). The cell supernatants were collected on day 14 after the treatment and investigated in accordance with the manufacturer's information.

The results are as follows:

### 1. Morphology of the BMMSC

Differences in the overall appearance of the cellular morphology were found depending on the culture medium. On day 1, both the BMMSC in the control medium and in the medium which included the extracts showed a spindle phenotype typical of cells obtained from bone marrow. On day 3, most of the cells in the control group had retained the spindle-like morphology. On day 7, many cells in the medium including the extracts exhibited a central accumulation with hypertrophic cells in their center. This indicated chondrocytes. The control groups retained their spindle shape throughout the period.

### 2. Characterization of the Mesenchymal Cells

The detection of cell surface antigens, so-called stromal stem cell markers, especially STRO-1, was positive in the control cells at passage 5. This showed the presence of human BMMSC.

### 3. The Cytotoxicity and the Proliferation

The investigations of the cytotoxicity were carried out to prove the cell-compatibility of the extracts on the cells. The results showed the good compatibility of the bovine joint and meniscus extracts on the BMMSC.

The detection of proliferation was carried out to investigate the growth of the human mesenchymal stem cells in the presence of two differently diluted solutions, 1:25 and 1:50. The cellular proliferation was evaluated on days 1, 3, 7, 14 and 21. Cell proliferation was found to be reduced in the cells treated with the joint and meniscus extracts by comparison with the control cells. This indicates cell differentiation. There was a significant increase in proliferation in untreated cells. In contrast to the proliferation rate of the cells treated with the joint and meniscus extracts, there was a graduated increase in the cell proliferation on treatment with TGF-β1, but maintaining a lower proliferation rate than that of the control groups. Since the bovine joint and meniscus extracts contain many of the growth factors and cytokines, this shows that the proliferation and differentiation may be based on factors other than TGF-β1.

### 4. Presence of TGF-β1 in the Extracts

Immunoprecipitation techniques prove the presence of TGF-β1 in the bovine joint and meniscus extracts.

### 5. Determination of Glycosaminoglycans (GAG)

Alcian blue and toluidine blue dyes form complexes with anionic glycoconjugates (AG) such as proteoglycans (PG) and glycosaminoglycans (GAG). Alcian blue was used to determine quantitatively the amount of chondroitin sulfate in cell lysates. Our results showed an increase in the amount of chondroitin sulfate in the cells treated with the bovine extracts by comparison with the untreated cells. It was further found that the cells treated with the bovine joint extracts contained more chondroitin sulfate than the cells treated with the bovine meniscus extracts. The parallel addition of 10 ng/ml TGF-β1 to the cultures demonstrated the increase in chondroitin sulfate. In addition, the toluidine blue stain was used for histological detection of glycosaminoglycans (GAG) in the BMMSC cultures in the presence of the joint and meniscus extracts. The results showed a clear blue cytoplasm with violet/purple granules, which proved the presence of GAG.

### 6. Absence of Calcium Deposits

The cells treated with solutions, diluted 1:25 and 1:50, of the bovine joint and meniscus extracts were analyzed using a light microscope. A drastic change in the morphology of treated cells was observed, with rounded shapes of the cells indicating hypertrophy and thus chondrocytic differentiation of the BMMSC. The untreated control cells retained their spindle shape. In addition, there were not signs of calcium deposits. The calcium deposits are evidence of an osteocytic differentiation, which was shown for BMMSC treated with bone matrix extracts, COLLOSS® and COLLOSS® E. The results thus prove that only a chondrocytic differentiation of the mesenchymal stem cells was induced by the extracts originally obtained from joint and meniscus.

### 7. Positive Expression of Cartilage-Specific Differentiation Markers

### a) SOX9

SOX9, a transcription factor which plays a key part in chondrogenesis, was significantly increased after treatment with the joint and meniscus extracts by comparison with the untreated control cells on day 14. These data demonstrate chondrogenesis.

### b) Aggrecan

Cartilage tissue includes aggrecan as one of its structural components. Aggrecan is a large chondroitin sulfate proteoglycan. It binds not only hyaluronan but also connecting proteins and, in this way, forms large aggregates. The aggrecan molecule is formed from a core protein and glycosaminoglycans, especially chondroitin sulfate. Aggrecan was detectable at the protein level by using mouse anti-aggrecan antibodies on day 14. Compared with untreated cells, the expression of aggrecan was very pronounced and localized in chondrocytically differentiated human mesenchymal stem cells. In addition, a stain was carried out only with secondary antibodies to ensure the specific binding of anti-aggrecan antibodies. Counting of aggrecan-positive cells demonstrated that the BMMSC treated with the bovine joint and meniscus extracts, as well as the BMMSC treated only with the recombinant TGF-β1, were present in larger numbers compared with the untreated control samples. However, the mRNA for aggrecan was reduced at the transcription level in treated cells in comparison with the untreated cells, but was significantly increased on treatment with TGF-β1. This might be attributable to the late expression of aggrecan. A further possibility is that further cytokines besides TGF-β1 are also involved in chondrogenesis.

8. Secretion of Osteopontin (OPN) and Collagen type I

The mesenchymal stem cells obtained from bone marrow and treated with bovine joint and meniscus extracts were free of secreted OPN in their supernatants. The levels of OPN in the treated cells were comparably high to those in the control cells. In addition, for comparison with the joint and meniscus extracts, COLLOSS® and COLLOSS® E were used in a solution diluted 1:50, as were BMP-2 and BMP-7 (OP1) with a concentration of 10 ng/ml. These extracts and recombinant proteins are known to be osteogenesis inducers and ought thus also to induce OPN production here. At the protein level, the type 1 collagen levels were similar with the mesenchymal stem cells treated with joint and meniscus extracts and with untreated mesenchymal stem cells. The differences were not significant. Although the tissues from which the joint and meniscus extracts were obtained were originally adjacent to bone tissue, there was no osteocytic differentiation of the mesenchymal stem cells after treatment with the joint and meniscus extracts. It is known by contrast that COLLOSS® and COLLOSS® E successfully induce differentiation of bone marrow-derived mesenchyrnal stem cells to osteocytes in vitro.

The bovine joint and meniscus extracts include a cocktail of cytokines and growth factors which belong to the superfamily of transforming growth factors (TGF). For this reason, 10 ng/ml TGF-β1 were used as a positive control for the induction of chondrogenesis in the cultures. The results show that TGF-β1 cannot be the only regulator of chondrogenesis. Induction of chondrogenesis depends on synergistic effects of many other growth factors and cytokines such as the bone growth factors (bone morphogenetic proteins, BMP). The BMP- and TGF-induced differentiation of BMMSC to cartilage or bone is concentration-dependent.

The cultivated BMMSC showed at passage 5 a characteristic elongate morphology (spindle shape) and expressed the cell surface marker STRO-1, which confirmed the presence of bone marrow stem cells. Treatment with solutions diluted 1:25 and 1:50, either of bovine joint extracts or of bovine meniscus extracts, induced characteristic morphological changes which started on day 7 and were maximally pronounced on day 14. The central accumulation with rounded cells which exhibited the hypertrophy phenotype typical of chondrocytes was observed. In the cultures treated with the extracts, this was linked to the decrease in the total cellular density, and to a greater extent in the cells treated with the meniscus extracts.

To prove that the joint and meniscus extracts induce a chondrocytic differentiation of BMMSC, the treated cells were investigated for the presence of chondro-specific markers, in particular SOX9, Aggrecan and GAG. The extracts induced a significant increase in SOX9 mRNA, expression of the chondro-specific cellular protein Aggrecan, and the presence of chondroitin sulfate in the cell lysates. This was supplemented by the toluidine blue stain for glycosaminoglycans (GAG), which was found to a copious extent in the cellular cytoplasm. The absence of osteopontin and collagen type I in the cellular supernatant confirmed that the bovine extracts lacked the property of inducing osteocytic differentiation of BMMSC. The absence of calcium deposition was detected in the H&E stain. COLLOSS®, COLLOSS® E, BMP-2 and BMP-7 (OP1) were used as positive controls of the induction of osteocytic differentiation. In summary, bovine extracts originally derived from joint and meniscus bring about chondrocytic differentiation of BMMSC, especially in vitro.

### Influence of Bone Marrow Extracts on the Number and Subtyping of Hematopoietic Stem Cell Colonies

### 1. Hematopoietic Cell Colony Assay

A CFC or CFU-C assay (colony-forming cell assay or colony-forming unit culture assay) was carried out as hematopoietic colony assay. The CFC assay is employed for counting hematopoietic progenitor cells. For this purpose, the progenitor cells are suspended in a mixture of culture medium, growth factors, and a semisolid matrix. The growth factors are usually cytokines. The growth factors promote the local expansion and differentiation of the hematopoietic cells into actual cell colonies. Methylcellulose or agarose for example is a suitable matrix. The suspended progenitor cells proliferate and differentiate and form colonies of blood cells which can be picked out with the aid of an inverting and dissecting microscope. In this connection, various subtypes of CFCs are differentiated:
CFU-macrophages (CFU-M)
CFU-granulocytes (CFU-G)
CFU-granulocytes/macrophages (CFU-GM)
Burst forming unit-erythroid (BFU-E)
CFU-granulocytes, macrophages, erythroid cells (CFU-GEMM): early multipotent progenitor cells.

### 2. Isolation of Bone Marrow Stem Cells and Activated Peripheral Blood Stem Cells

The bone marrow stem cells were taken from 5 patients. The peripheral blood stem cells were taken by apheresis from a healthy donor and activated with G-CSF for 5 days. 4 ml portions of the stem cell samples obtained in this way were diluted and washed with 4 ml of a Hank's saline solution, shaking gently several times. The washed stem cell samples were then layered on 5 ml of a Ficoll solution. The mixtures were centrifuged (200 g or 1400 rpm) at room temperature in each case for 30 min. The mononuclear cell layer was then removed and washed with Hank's saline solution. The cells were centrifuged at 800 rpm for 10 min and resuspended in a culture medium of IMD methylcellulose. The cells were subsequently aliquoted in duplicates of 1×10⁵ cells per plate and incubated in the presence or absence of bone marrow extracts. The incubation was carried out at 37° C. in a humid incubator with 5% CO₂ with minimal disturbance. After 14 to 18 days, the plates were counted for CFUs (colony-forming units) in accordance with standard criteria.

### 3. Methylcellulose-Based Culture Media for the Human CFC Assay

The following culture media were employed:
HSC001: Methylcellulose stock solution (free of FBS (fetal bovine serum) or cytokines).
HSC002: Human medium based on methylcellulose which contains FBS, BSA (bovine serum albumin), L-Gln (L-glutamine) and 2-mercaptoethanol. The components were optimized for the CFC assay.
HSC003: Human medium completely composed of methylcellulose which contains FBS, BSA (bovine serum albumin), L-Gln, (L-glutamine) and 2-mercaptoethanol, recombinant human SCF, recombinant human G-CSF, recombinant human IL-3 and recombinant human erythropoietin (Epo). The components were optimized for the CFC assay and assist the growth of burst-forming and colony-forming erythroid cells (BFU-E, CFU-E), myeloid cells (CFU-GM, CFU-G, CFU-M) and cell colonies of mixed lineage (CFU-GEMM).
HSC004: Medium completely composed of methylcellulose without Epo, which comprises FBS, BSA (bovine serum albumin), L-Gln (L-glutamine) and 2-mercaptoethanol, recombinant human SCF, recombinant human G-CSF and recombinant human IL-3. The components were optimized for the CFC assay and assist the growth of myeloid cell colonies (CFU-GM, CFU-G and CFU-M).
HSC005: Human medium enriched with methylcellulose which contains EBS, BSA (bovine serum albumin), L-Gln (L-glutamine) and 2-mercaptoethanol, recombinant human SCF, recombinant human G-CSF, recombinant human IL-3, recombinant human erythropoietin (Epo) and recombinant human IL-6. The components were optimized for the CFC assay and assist the growth of a purified population of colony-forming erythroid cells (BFU-E, CFU-E), myeloid cells (CFU-GM, CFU-G, CFU-M) and of mixed cell lines (CFU-GEMM).

### 4. Detailed Instructions for the Method for the CFC Assay in Methylcellulose-Based Media

The vessels with the methylcellulose medium (HSC001, HSC002, HSC003, HSC004 or HSC005) were thawed. Bone marrow extracts were then added in a concentration of 0.0125 mg/ml and stem cells were added (1×10⁵ cells per plate) in a volume ratio in duplicates of 1:10. The cells were thoroughly mixed. Subsequently, 1.5 ml thereof were in each case dispersed in 35 mm culture plates in duplicates. Thereafter, two plates with the samples and one uncovered water plate were placed in a 100 mm culture plate and covered. The cells were incubated in the presence and absence of the bone marrow extracts (various solutions) at 37° C. in a humid incubator with 5% CO₂ without disturbance.

The cells proliferated and differentiated into single colonies during incubation for 14 to 18 days.

The colonies were then counted and characterized according to their morphology. An inverted microscope and a 100 mm culture plate marked with a selection grid were used for this purpose. To investigate the individual cell morphology, single cell colonies were picked up with a 200 μl pipette. This was achieved by loading a clean pipette with about 80 μl of IMDM and then harvesting the colony from the semisolid medium with the aid of the microscope. The cells were then distributed on glass slides using Cytospin and stained with the Wright-Giemsa stain.

### 5.1 Morphological Results

Investigation of the cell morphology of cell colonies which were cultured in the HSC002 medium clearly revealed the morphology of monocytes and macrophages after Wright-Giemsa staining.

5.2 Effects of the Bone Marrow Extracts (Including COLLOSS® E) on the Number and Subtypes of Hematopoietic Stem Cell Colonies from Bone Marrow

No colonies of hematopoietic stem cells were observable in the HSC001 culture medium.

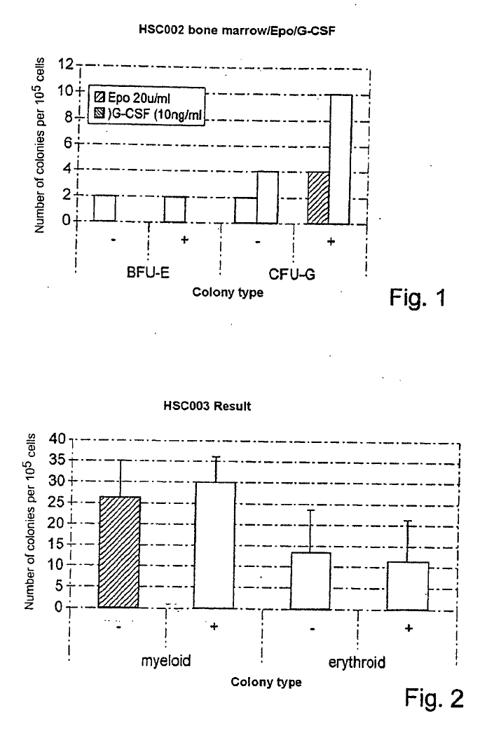

By contrast, addition of bone marrow extracts to the HSC002 medium led to induction of the growth of myeloid colonies in the presence of FBS alone. Further, addition of bone marrow extracts to G-CSF-induced myeloid cell colonies (CFU-G: colony-forming unit-granulocyte) led to a significant increase both in the number and in the size of the colonies (FIG. 1: +/− means in the presence of bone marrow extract/in the absence of bone marrow extract). By contrast, addition of bone marrow extracts to HSC002 did not bring about an increase in the number of Epo-stimulated erythroid colonies.

Moreover, the added bone marrow extracts to the HSC003 medium did not lead to any noticeable changes in the growth of myeloid or erythroid cell colonies (FIG. 2: +/− means in the presence of bone marrow extract/in the absence of bone marrow extract). This is probably attributable to the presence of overlapping cytokines between the HSC003 medium and the bone marrow extracts. However, the HSC003 medium might also mask the effects of the bone marrow extracts. The colony-forming cells which grew in the presence or absence of bone marrow extracts were, however, mostly of myeloid origin.

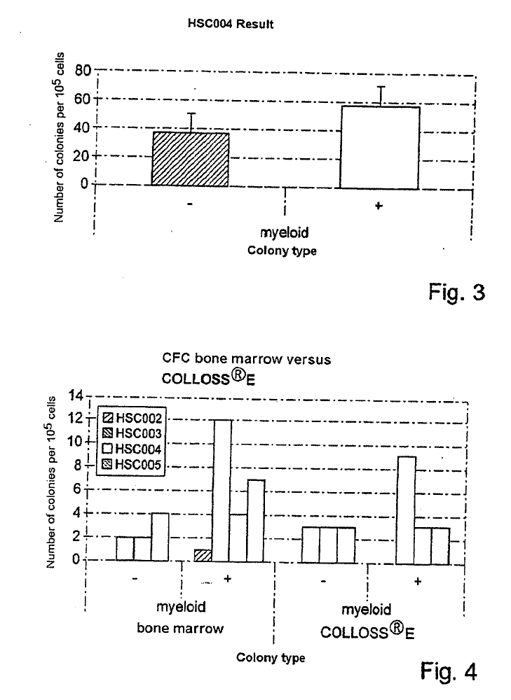

Addition of bone marrow extracts to the HSC004 medium brought about overall (and for each individual sample) a marked increase in growth of colony-forming cells with myeloid potential (FIG. 3: +/− means in the presence of bone marrow extract/in the absence of bone marrow extract).

Addition of bone marrow extracts to the HSC005 medium led to no noticeable changes in the growth of colony-forming cells with myeloid or erythroid potential. This observation is also probably attributable to the presence of overlapping cytokines between the HSC005 medium and the bone marrow extracts. However, the HSC005 medium might likewise also mask the effects of the added bone marrow extracts. The colony-forming cells which grew in the presence or absence of bone marrow extracts were, however, mostly of myeloid origin.

Moreover, bone marrow extracts showed by comparison with the bone extract COLLOSS® E (a product which is explained in detail in the description) showed a higher growth potential of colony-forming cells. However, COLLOSS® E brought about an increased formation of myeloid colonies in HSC003 (FIG. 4: +/− means in the presence of bone marrow extract/in the absence of bone marrow extract). The concentration of COLLOSS® E was 0.14 mg/ml and was thus ten times higher than the concentration of the bone marrow extracts used (0.0125 mg/ml).

5.3 Effects of the Bone Marrow Extracts on the Number and Subtypes of Hematopoietic Stem Cell Colonies which are Derived from Activated Peripheral Blood Stem Cells.

Myeloid and erythroid cell colonies were observed in the presence and absence of bone marrow extracts.

Addition of bone marrow extracts to the HSC002 medium did not lead to a significant increase in the growth of myeloid cell colonies.

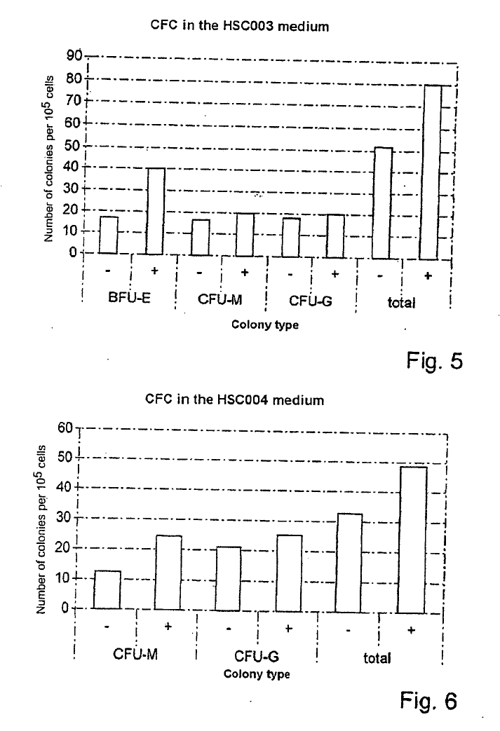

However, after addition of bone marrow extracts to the HSC003 medium it was possible to find a significantly increased formation of erythroid cell colonies (BFU-E: burst forming unit-erythroid) and overall a distinctly increased formation of cell colonies in HSC003 (FIG. 5: +/means in the presence of bone marrow extract/in the absence of bone marrow extract).

In addition, the bone marrow extracts brought about in the HSC004 medium a distinctly increased growth of colony-forming cells with myeloid potential (FIG. 6: CFU-M: colony-forming unit-macrophage; +/− means in the presence of bone marrow extract/in the absence of bone marrow extract). Addition of bone marrow extracts to HSC004 additionally caused a distinct increase in the colony-forming cells overall.

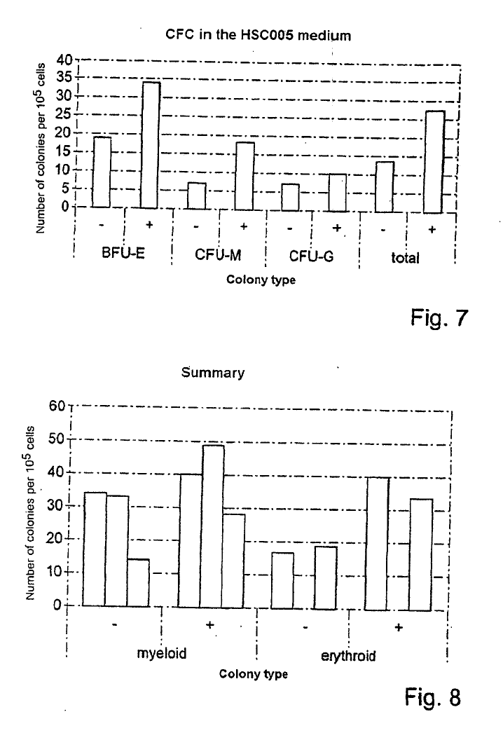

It was likewise possible after addition of the bone marrow extracts to the HSC005 medium to find a significantly increased formation of erythroid and myeloid cell colonies (FIG. 7: BFU-E: burst forming unit-erythroid and CFU-M: colony-forming unit-macrophage; +/−means in the presence of bone marrow extract/in the absence of bone marrow extract).

In summary, it is therefore possible to say that starting from activated peripheral blood stem cells the formation of cell colonies with a myeloid lineage is increased in the HSC004 and HSC005 media. It is additionally possible to find an increased formation of cell colonies with erythroid lineage in the HSC003 and HSC005 media after addition of the bone marrow extracts (FIG. 8: +/− means in the presence of bone marrow extract/in the absence of bone marrow extract).

Treatment of Mesenchymal Stem Cells from Bone Marrow with a Pancreas Extract

After treatment of mesenchymal stem cells from bone marrow of a patient with a pancreas extract it was possible to detect the expression of Glut2 in the treated stem cells. Glut2 is a specific transporter protein of the β cells of the pancreas.

1. A method for at least partially differentiating stem cells and/or progenitor cells to at least one tissue type, comprising:
treating the stem cells and/or the progenitor cells with extracts which include active substances and/or components for differentiating stem cells and/or progenitor cells, and
cultivating and differentiating the stem cells and/or the progenitor cells.

2. The method as claimed in claim 1, Wherein embryonic, fetal, neonatal, adult (somatic) stem cells and/or progenitor cells are used for the treatment.

3. The method as claimed in claim 1, wherein the stem cells and/or progenitor cells exhibit, after treatment with the extracts, on cell surface specific molecules and/or a morphology characteristic of particular tissues.

4. The method as claimed in claim 1, wherein extracts which are derived from organic materials are employed for the treatment.

5. The method as claimed in claim 1, wherein extracts which are of human and/or animal origin are employed for the treatment.

6. The method as claimed in claim 1, wherein extracts which are of vegetable and/or microbial origin are employed for the treatment.

7. The method as claimed in claim 1, wherein extracts which have been obtained from cells and/or tissues are employed for the treatment.

8. The method as claimed in any claim 1, wherein extracts which are derived from bone and/or cartilage, muscle, tendon, ligament, lung, blood, skin, liver, kidney, glandular tissue, brain and/or nerve tissue, and are bovine, equine and/or human origin, are employed for the treatment.

9. A method of producing extracts in which organic materials are subjected to at least one denaturation operation for extraction comprising providing cells and/or tissues as organic materials which include active substances and/or components which activate or inhibit, in particular activate, the growth and/or differentiation of stem cells and/or progenitor cells to at least one tissue type.

10. The method as claimed in claim 9, wherein the organic materials are of human and/or animal origin.

11. The method as claimed in claim 9, wherein the organic materials are of vegetable or microbial origin.

12. The method for producing extracts as claimed in claim 9, wherein the organic materials take the form of cartilage, muscle, in tendon, ligament, lung, blood, skin, liver, kidney, glandular tissue, brain or nerve tissue, and are bovine, equine and/or human origin.

13. An extract produced according to the method of claim 9, comprising active substances and/or components (which are not derived from bone) which activate or inhibit, the growth and/or differentiation of stem cells and/or progenitor cells to at least one tissue type.

14. The extract as claimed in claim 13, wherein the active substances and/or the components comprise at least leukotrienes, cytotactin, tenascin, laminin, fibronectin, cytokines, and collagen.

15. The extract as claimed in claim 13, wherein the active substances and/or components are present in combination, and in a complex.

16-19. (canceled)

20. A method for differentiating stem cells and/or progenitor cells, comprising subjecting undifferentiated cells and/or incompletely differentiated cells to a treatment with extracts wherein the extracts have been obtained from cells and/or tissues.
